(19) United States
(12) Patent Application Publication
(10) Pub. No.: US 2024/0092954 A1
(43) Pub. Date: Mar. 21, 2024

(54) POLYMER THIN FILM FOR CAPTURING AND RELEASING NUCLEIC ACID WITH HIGH EFFICIENCY AND METHOD FOR EXTRACTING NUCLEIC ACID USING THE SAME

(71) Applicant: Korea Advanced Institute of Science and Technology, Daejeon (KR)

(72) Inventors: Sung Gap IM, Daejeon (KR); Seongeun KIM, Daejeon (KR); Boo Seok JEONG, Daejeon (KR); Nahyun PARK, Daejeon (KR); Younseong SONG, Daejeon (KR); Hogi KIM, Daejeon (KR)

(73) Assignee: Korea Advanced Institute of Science and Technology, Daejeon (KR)

(21) Appl. No.: 18/452,831

(22) Filed: Aug. 21, 2023

(30) Foreign Application Priority Data
Aug. 25, 2022 (KR) 10-2022-0106759

(51) Int. Cl. C08F 220/34

(52) U.S. Cl. CPC C08F 220/34; C12N 15/1006

(57) ABSTRACT

A disclosed polymer thin film includes a cationic polymer having a hydrolyzable side chain. The polymer thin film has a surface potential of 1 mV to 50 mV. A hydrolysis efficiency of the cationic polymer is 30% or more after reaction at 50° C. for one hour. The cationic polymer is dissolved to three-dimensionally capture nucleic acids, and releases the nucleic acids through hydrolysis. Thus, nucleic acid capturing-releasing efficiency can be improved.

### CROSS-REFERENCE TO RELATED APPLICATION

This application claims priority under 35 USC § 119 to Korean Patent Application No. 10-2022-0106759 filed on Aug. 25, 2022 in the Korean Intellectual Property Office (KIPO), the entire disclosure of which is incorporated herein by reference.

### BACKGROUND

### 1. Field

The present disclosure relates to a polymer thin film for extracting nucleic acids. More specifically, the present disclosure relates to a polymer thin film for capturing and releasing nucleic acids with high efficiency and a method for extracting nucleic acids using the same.

### 2. Description of the Related Art

The recent spread of highly contagious and toxic epidemics has threatened the world's health and economy. Accordingly, there has been a surge in demand for technology which quickly and accurately diagnoses diseases in order to prevent the spread of infectious diseases and reduce damage. In the case of diseases caused by viruses, rapid diagnosis is a more essential factor because the diseases lead to many variants due to the properties of the virus in which mutations frequently occur. Currently, reverse transcription quantitative PCR (RT-qPCR) is frequently used as a diagnostic method, and is capable of diagnosing various diseases by amplifying various pathogen genes as well as coronavirus.

A molecular diagnostic process consists of sample collection, nucleic acid extraction, target gene amplification, and detection processes. In particular, the nucleic acid extraction process is complicated (viral lysis, viral RNA or DNA capture in a solid matrix, washing, and release), and thus consumes a lot of time and reagents. An important step in the nucleic acid extraction process is nucleic acid capture and release (elution). For accurate and rapid diagnosis, it is necessary to capture and release nucleic acid with high efficiency before the amplification step. A nucleic acid capturing method using silica, magnetic beads, or the like, which is currently known, may achieve relatively high capturing efficiency, but has low elution efficiency. As methods for increasing the elution efficiency of nucleic acid, a strategy of using chaotropic reagents or increasing pH or temperature is used, but the methods may adversely affect the efficiency of a subsequent amplification process and have low compatibility.

### SUMMARY

One object of the present disclosure is to provide a polymer thin film for capturing and releasing nucleic acids (DNA or RNA) with high efficiency.

Another object of the present disclosure is to provide a method for extracting nucleic acids using the polymer thin film.

However, the objects of the present disclosure are not limited to the objects described above, and may be variously expanded without departing from the spirit and scope of the present disclosure.

A polymer thin film according to an embodiment of the present disclosure includes a cationic polymer having a hydrolyzable side chain. The polymer thin film has a surface potential of 1 mV to 50 mV. A hydrolysis efficiency of the cationic polymer is 30% or more after reaction at 50° C. for one hour.

In an embodiment, the hydrolyzable side chain includes a hydrolytic cationic ester.

In an embodiment, the cationic polymer includes a repeating unit polymerized from a monomer including at least one of 2-(dimethylamino)ethyl acrylate (DMAEA), 2-(dimethylamino)ethyl methacrylate (DMAEMA), and 2-(diethylamino)ethyl methacrylate (DEAEMA).

In an embodiment, the cationic polymer includes a first repeating unit polymerized from a first monomer including the hydrolytic cationic ester and a second repeating unit polymerized from a second monomer different from the first monomer. The second monomer includes at least one of 4-vinylpyridine (4VP), vinylimidazole (VIDZ), dimethylaminomethylstyrene (DMAMS), vinylbenzylchloride (VBC), chloroethylacrylate (CEA), divinylbenzene (DVB), glycidylmethacrylate (GMA), di(ethyleneglycol)divinylether (DEGDVE), ethyleneglycoldimethacrylate (EGDMA), ethyleneglycoldiacrylate (EGDA), 1,4-butadienedioldiacrylate (BDDA), 2,4,6,8-tetramethyl -2,4,6,8-tetravinylcyclotetrasiloxane (V4D4), 1,3,5 -trimethyl -1,3,5-trivinyl-cyclotrisiloxane (V3D3), and cyclohexyl methacrylate (CHMA).

In an embodiment, a molar ratio of the first repeating unit and the second repeating unit is 1:0.1 to 1:0.3.

In an embodiment, the cationic polymer includes a repeating unit polymerized from DMAEA.

In an embodiment, the cationic polymer is a linear polymer.

In an embodiment, the cationic polymer has a branched or cross-linked structure.

In an embodiment, the cationic polymer has a number average molecular weight of 1,000 g/mol to 3,000 g/mol.

A method for extracting nucleic acid includes dissolving a cationic polymer having a hydrolyzable side chain to combine the dissolved cationic polymer with nucleic acid by electrostatic attraction, precipitating a polyplex formed by combination of the cationic polymer with the nucleic acid, and hydrolyzing the cationic polymer to release the nucleic acid from the polyplex.

In an embodiment, hydrolyzing the cationic polymer to release the nucleic acid is performed at 30° C. to 60° C.

In an embodiment, the cationic polymer is combined with the nucleic acid at a temperature of 10° C. or more and less than 30° C.

In an embodiment, the hydrolyzed side chain of the cationic polymer has a negative charge.

As described above, according to exemplary embodiments of the present disclosure, nucleic acids may be three-dimensionally captured using a dissolved cationic polymer in a capturing step for extracting nucleic acids, thereby expanding a region for polymer-nucleic acid interaction. Thus, nucleic acid capturing efficiency can be enhanced.

In addition, the nucleic acids can be released by electrostatic repulsion between the polymer and the nucleic acids generated by the self-catalyzed hydrolysis of the polymer. Thus, more nucleic acids can be released at a temperature lower than that of a conventional method for releasing nucleic acids, without changing conditions (such as pH 10, heating (e.g., to 95° C.), addition of chaotropic reagents, or the like) which can affect an amplification step.

In addition, the polymer can have stability in the amplification step subsequent to a nucleic acid extraction step. Accordingly, it can be possible to implement a detection system for continuously performing the nucleic acid extraction step and the amplification step.

### BRIEF DESCRIPTION OF THE DRAWINGS

Example embodiments can be understood in more detail from the following description taken in conjunction with the accompanying drawings, in which:

FIG. 1 is a sectional view showing a container for extracting nucleic acid including a polymer thin film according to an embodiment of the present disclosure.

FIGS. 2A, 2B, 2C, and 2D are enlarged sectional views showing region A of FIG. 1 to explain a method for extracting nucleic acids according to an embodiment of the present disclosure.

FIG. 4 is a graph showing the results of a gel permeation chromatography (GPC) analysis of a molecular weight of the DMAEA polymer of Example 1.

FIG. 5 is a graph showing hydrolysis efficiency (%) over times (five-minute intervals) by subjecting the DMAEA polymer of Example 1 to hydrolysis at pH 8.0, 25° C. and 50° C.

FIG. 6 is a graph showing a zeta-potential before and after hydrolysis of the DMAEA polymer of Example 1.

FIG. 7 is a graph showing a zeta-potential according to a ratio of DMAEA polymer of Example 1 and RNA (nitrogen (N)/phosphate (P)).

FIG. 8 is a graph showing an average hydrodynamic diameter (Z-average) according to a ratio of DMAEA polymer and RNA of Example 1 (nitrogen (N)/phosphate (P)).

FIG. 9 is a graph showing the capture efficiency according to a centrifugation time in the nucleic acid capturing experiment using the DMAEA polymer of Example 1.

FIG. 10 is a graph showing capture efficiency during a centrifugation time optimized in a nucleic acid capturing experiment using the polymers of Example 1 (pDMAEA), Example 2 (pDV1), and Example 3 (pDV2).

FIG. 11 is a graph showing the recovery efficiency in a nucleic acid extraction experiment using the polymers of Example 1 (pDMAEA), Example 2 (pDV1), and Example 3 (pDV2).

### DETAILED DESCRIPTION OF THE EXEMPLARY EMBODIMENTS

Various embodiments will be described more fully hereinafter with reference to the accompanying drawings, in which some embodiments are shown. The present invention may, however, be embodied in many different forms and should not be construed as limited to the embodiments set forth herein. Rather, these embodiments are provided so that this description will be thorough and complete, and will fully convey the scope of the present invention to those skilled in the art. In the drawings, the sizes and relative sizes of layers and regions may be exaggerated for clarity.

It will be understood that when an element or layer is referred to as being “on,” “connected to” or “coupled to” another element or layer, it can be directly on, connected or coupled to the other element or layer or intervening elements or layers may be present. In contrast, when an element is referred to as being “directly on,” “directly connected to” or “directly coupled to” another element or layer, there are no intervening elements or layers present. Like numerals refer to like elements throughout. As used herein, the term “and/or” includes any and all combinations of one or more of the associated listed items.

It will be understood that, although the terms first, second, third etc. may be used herein to describe various elements, components, regions, layers and/or parts, these elements, components, regions, layers and/or parts should not be limited by these terms. These terms are only used to distinguish one element, component, region, layer or part from another region, layer or part. Thus, a first element, component, region, layer or part discussed below could be termed a second element, component, region, layer or part without departing from the teachings of the present invention.

The terminology used herein is for the purpose of describing particular embodiments only and is not intended to be limiting of the present invention. As used herein, the singular forms “a,” “an” and “the” are intended to include a plurality of forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

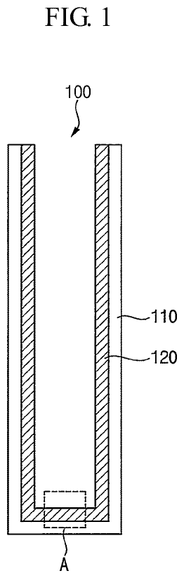

FIG. 1 is a sectional view showing a container for extracting nucleic acid including a polymer thin film according to an embodiment of the present disclosure.

Referring to FIG. 1, a container for extracting nucleic acid 100 may include a body 110 and a polymer thin film 120.

According to an embodiment, the container for extracting nucleic acid 100 may have a tube shape with one side open. However, the embodiments of the present disclosure are not limited thereto, and the container for extracting nucleic acid 100 may have various shapes capable of receiving a sample, such as a well plate, etc.

The body 110 may receive a sample including nucleic acids. In addition, the body 110 may serve as a substrate for supporting the polymer thin film 120. For example, the polymer thin film 120 may be coated on an inner wall of the body 110. According to an embodiment, the polymer thin film 120 may entirely cover the inner wall of the body 110. However, the embodiments of the present disclosure are not limited thereto, and the polymer thin film 120 may be disposed on a portion of the inner wall of the body 110, for example, on a bottom surface.

For example, the body 110 may include polypropylene (PP), polyethylene (PE), polyvinyl chloride (PVC), polycarbonate (PC), polyester, polymethylmethacrylate (PMMA), polyethylene terephthalate (PET), polyurethane (PU), silicon, glass, quartz, ceramic, Teflon, or a combination thereof.

The polymer thin film 120 may include a polymer. The polymer may be defined to include an oligomer and/or a polymer having a molecular weight greater than that of the oligomer. The polymer may be a homopolymer having the same repeating unit or a copolymer having two or more different repeating units.

The polymer thin film 120 may include a cationic polymer, and a surface thereof may have a positive potential. For example, a surface potential of the polymer thin film 120 may be 1 mV to 50 mV, but is not limited thereto, and may preferably be 10 mV or more, for example, 10 mV to 40 mV.

For example, the polymer may include a repeating unit having positively charged nitrogen in a side chain. According to an embodiment, the polymer may include at least a repeating unit having a tertiary amine at an end of the side chain.

The polymer thin film 120 may include a hydrolyzable polymer. For example, the polymer may have a hydrolyzable side chain. A hydrolysis efficiency (%) of the polymer may be defined as a ratio of the hydrolyzed repeating unit to the repeating unit of the entire polymer, and may be 30% or more after reaction at 50° C. for one hour. The higher the hydrolysis efficiency, the higher the nucleic acid release efficiency of the polymer, and the shorter the hydrolysis time, the shorter the time required for extracting nucleic acids. Preferably, the hydrolysis efficiency of the polymer may be 30% to 100% after reaction at 50° C. for 30 minutes, more preferably, 30% or more after reaction at 50° C. for five minutes. For example, the hydrolysis efficiency may be measured at about pH 8. The pH condition may be intended to provide a reference for measuring the hydrolysis efficiency, and thus the condition of the present disclosure is not limited thereto, and the polymer of the present disclosure may be hydrolyzed in various pH ranges.

The hydrolysis efficiency of the polymer may vary depending on a structure of the polymer, a molecular weight of the polymer, chemical properties of the side chain, steric hindrance due to the side chain, and the like.

According to an embodiment, the polymer may be a linear polymer. Since the linear polymer has high solubility and a large amount of hydrolyzable side chains, the capture-release efficiency of nucleic acids may be improved. According to another embodiment, the polymer may have a branched or cross-linked structure. When the polymer has a branched or cross-linked structure, a molecular weight may increase and a precipitation rate may increase in a centrifugation step, thereby reducing a total of time required for extracting nucleic acids.

For example, the polymer may have a number average molecular weight of about 1,000 to about 3,000. When the molecular weight of the polymer is excessively small, a size of a polyplex formed by combining the polymer with nucleic acids is too small, and thus may be difficult to be separated and purified. In addition, when the molecular weight of the polymer is excessively large, solubility may increase and hydrolysis efficiency may decrease, thereby reducing the capture-release efficiency of nucleic acids. When the polymer has a branched or cross-linked structure, the polymer may have a molecular weight greater than that of a linear polymer. For example, the linear polymer may have a number average molecular weight of 1,000 to 2,000, and the polymer having a branched or cross-linked structure may have a number average molecular weight of 1,500 to 3,000. However, the embodiments of the present disclosure are not limited thereto, and may have a molecular weight in various ranges within which appropriate hydrolysis efficiency may be achieved.

According to an embodiment, the polymer may include a hydrolytic cationic ester group as a side chain. The hydrolytic cationic ester group may be capable of self-catalyzed hydrolysis by an intramolecular interaction between a cationic group (e.g., dialkylamino group) and an ester carbonyl group.

For example, the polymer may include poly[2-(dimethylamino)ethyl acrylate] (p-DMAEA), poly[2-(dimethylamino)ethyl methacrylate (p-DMAEMA), poly[2-(diethylamino)ethyl methacrylate (p-DEAEMA), or the like. For example, the polymer may include the following repeating unit.

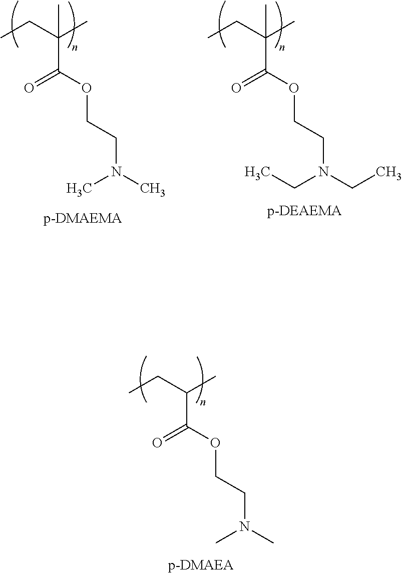

According to an embodiment, the polymer may preferably include p-DMAEA in terms of hydrolysis efficiency. A tertiary amine group (dialkylamino group) of p-DMAEA may have an alkyl group having a relatively small size, and may have no steric hindrance due to a methyl group bonded to a main chain, and thus may easily interact with an ester carbonyl group.

For example, when the polymer is a copolymer, it may be obtained by copolymerization between a first monomer having a hydrolytic cationic ester as a side chain and a second monomer different from the first monomer.

For example, the first monomer may include DMAEA, DMAEMA, DEAEMA, or a combination thereof

The second monomer may include a nucleophile monomer, a cross-linkable monomer, or a combination thereof. The nucleophile monomer may include a vinyl group, an acrylic group, or a methacrylic group, and a tertiary amine group. The cross-linkable monomer may include at least two of the vinyl group, the acrylic group, or the methacrylic group, or may include the vinyl group, the acrylic group, or the methacrylic group, and an alkyl halide group.

For example, the second monomer may include 4-vinylpyridine (4VP), vinylimidazole (VIDZ), dimethylaminomethylstyrene (DMAMS), vinylbenzylchloride (VBC), chloroethylacrylate (CEA), divinylbenzene (DVB), glycidylmethacrylate (GMA), di(ethyleneglycol)divinylether (DEGDVE), ethyleneglycoldimethacrylate (EGDMA), ethyleneglycoldiacrylate (EGDA), 1,4 -butadienedioldiacrylate (BDDA), 2,4,6, 8-tetramethyl-2,4,6, 8-tetravinylcyclotetrasiloxane (V4D4), 1,3,5-trimethyl-1,3,5-trivinyl-cyclotrisiloxane (V3D3), cyclohexylmethacrylate (CHMA), or a mixture thereof. For example, when the second monomer includes a cross-linkable monomer such as vinylbenzyl chloride, chloroethylacrylate, divinylbenzene, glycidylmethacrylate, di(ethyleneglyycol)divinylether, ethyleneglycoldimethacrylate, ethyleneglycoldiacrylate, 1,4-butadienedioldiacrylate, 2,4,6,8 -tetramethyl-2,4,6,8-tetravinylcyclotetrasiloxane, 1,3,5-trimethyl-1,3,5-trivinylcyclotrisiloxane, and the like, the copolymer may have a cross-linked structure.

For example, in the copolymer, a molar ratio of the first repeating unit derived from the first monomer and the second repeating unit derived from the second monomer may be 1:0.1 to 1:0.3. When the ratio of the second monomer is excessively high, the hydrolysis efficiency may decrease and thus the nucleic acid release efficiency may deteriorate.

For example, a thickness of the polymer thin film 120 may be 1 nm to 1,000 nm, or 10 nm to 500 nm, but is not limited thereto, and may be adjusted according to a use, a type of sample, and the like.

For example, the polymer thin film 120 may be formed on a substrate by a chemical vapor deposition method. For example, the vaporized monomer may be activated in a reactor to perform a polymerization reaction, and thus the polymer thin film may be coated on the substrate. For example, the substrate may be the body 110. However, the embodiments of the present disclosure are not limited thereto, and the polymer thin film may be coated on a separate substrate and then disposed together with the substrate in the body 110.

According to an embodiment, the polymer thin film 120 may be formed by initiated chemical vapor deposition (iCVD) using an initiator. According to iCVD, the vaporized initiator and monomer may be adsorbed to a surface of the substrate. The initiator may be activated by heating by a filament or ultraviolet rays. A chain polymerization reaction may be initiated by free radicals generated by an activated initiator. Thus, the polymer thin film may be formed on the substrate. The method may not use a solvent or the like, and synthesis and coating may be performed in one step. Thus, the uniformity of the polymer may increase, and process variables may be adjusted to form a thin film having a desired thickness.

For example, a peroxide such as tert-butyl peroxide (TBPO) may be used as the initiator. However, the embodiments of the present disclosure are not limited thereto, and a benzophenone-based initiator which may be activated by UV may be also used.

The polymer thin film according to embodiments of the present disclosure may include a cationic polymer and may have high solubility in water. Thus, the polymer may capture nucleic acids in a dissolved state during a nucleic acid extraction process. Accordingly, a capturing area may greatly increase, thereby increasing capturing efficiency and shortening a capturing time.

In addition, the polymer may be hydrolyzed by autocatalysis. Thus, a more amount of nucleic acids may be released at a temperature lower than a temperature in conventional methods of releasing nucleic acids by heating.

For example, a container for extracting nucleic acid having the polymer thin film may be used for pretreatment (nucleic acid extraction step) of polymerase chain reaction (PCR). However, the embodiments of the present disclosure are not limited thereto, and a tube for extracting nucleic acid may be also used as a container for an amplification step after the nucleic acid extraction step.

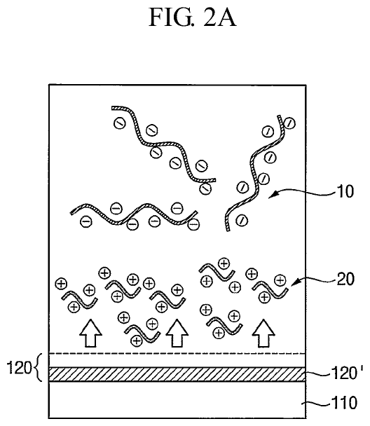

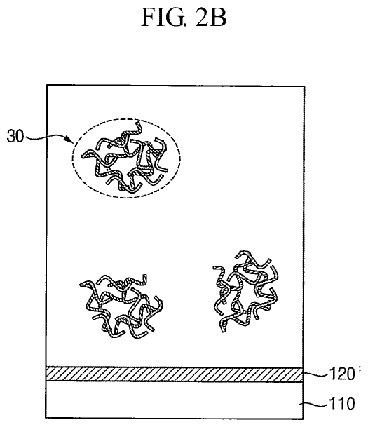

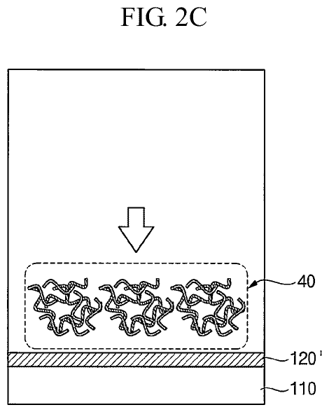

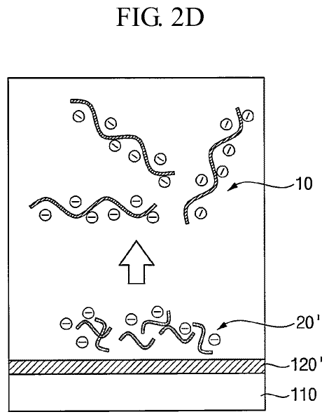

A nucleic acid capture-release mechanism of the polymer will be described in detail with reference to a method for extracting nucleic acids according to an embodiment of the present disclosure. FIGS. 2A, 2B, 2C, and 2D are enlarged sectional views showing region A of FIG. 1 to explain a method for extracting nucleic acids according to an embodiment of the present disclosure.

Referring to FIGS. 2A and 2B, a sample including nucleic acid 10 may be provided in the container for extracting nucleic acid. The container for extracting nucleic acid may include a polymer thin film 120 disposed therein. The polymer thin film 120 may include a cationic polymer and have a positive surface potential. The polymer may be easily dissolved by water or the like. The dissolved polymer 20 from the polymer thin film 120 may have a positive charge, and the nucleic acid 10 may have a negative charge. Accordingly, the dissolved polymer 20 may capture the nucleic acid 10 by electrostatic attraction. The dissolved polymer 20 may be bound to the nucleic acid 10 by electrostatic attraction to form a composite 30 of a polymer and a nucleic acid, which may be referred to as a polyplex or a polymer-nucleic acid composite, hereinafter.

According to the embodiments of the present disclosure, a region where electrostatic binding between the polymer and the nucleic acid occurs is not limited to a two-dimensional surface of the polymer thin film 120, but may extend to a three-dimensional interface between the dissolved polymers 20 and a sample. Thus, the sampling efficiency and the capturing speed of nucleic acids may greatly increase.

For example, the nucleic acid 10 may include ribonucleic acid (RNA), deoxyribonucleic acid (DNA), methylphosphonate nucleic acid, phosphothioate oligonucleotide (S-oligo), c-DNA, miRNA, aptamer, and the like.

A sample including the nucleic acid 10 may be derived from a biological organism. For example, the sample may include a biological fluid. The biological fluid may include urine, blood, plasma, serum, saliva, semen, stool, sputum, cerebrospinal fluid, tears, mucus, amniotic fluid, and the like. In addition, the sample may include a biological tissue of humans, animals, plants, bacteria, fungi, and viruses, a cell thereof, or an intracellular material thereof. Furthermore, the sample may include an environmental sample such as drinking water, food, etc.

For example, the polymer may be dissolved by the sample. However, embodiments of the present disclosure are not limited thereto. For example, the polymer may be dissolved by a buffer for dissolving nucleic acids or a sample mixed with the buffer.

The polymer thin film 120 may be entirely or partially dissolved. For example, a portion 120′ of the polymer thin film may remain on the body 110 of the extraction container.

The nucleic acid capturing step described above may be performed at room temperature. For example, the steps may be performed at 10° C. or more and less than 30° C., preferably at 20° C. or more and less than 30° C. When a temperature of the nucleic acid capturing step is excessively high, the hydrolysis of the polymer may proceed, and the nucleic acid capturing efficiency may decrease.

Referring to FIG. 2C, a polymer-nucleic acid composite may be precipitated. In order to precipitate the polymer-nucleic acid composite, centrifugation may be performed. For example, as adjacent polymer-nucleic acid composites aggregate, a precipitate 40 may be formed by the aggregated polymer-nucleic acid composites.

For example, after the precipitation of the polymer-nucleic acid composite, the supernatant may be removed to remove impurities such as suspended materials, etc. After removing the supernatant, the precipitate 40 may be washed again by using a washing buffer, purified water, or the like, in order to further get rid of the foreign substances. Preferably, the precipitation and washing of the polymer-nucleic acid composite may be performed in-situ in the container for extracting nucleic acid.

Referring to FIG. 2D, nucleic acid 10 may be released from the precipitated polymer-nucleic acid composite. According to embodiments of the present disclosure, hydrolysis may be used to release the nucleic acid 10.

The polymer may be hydrolyzed by autocatalysis (self-catalysis). For example, the repeating unit of the polymer may have a hydrolytic cationic ester group as a side chain. Self-catalyzed hydrolysis may proceed by an intramolecular interaction between a cationic group (e.g., dialkylamino group) and an ester carbonyl group. For example, when the polymer is p-DMAEA, it may be hydrolyzed as follows to be converted into a copolymer of DMAEA and acrylic acid (AA), and dimethylaminoethanol may be produced as a reaction by-product.

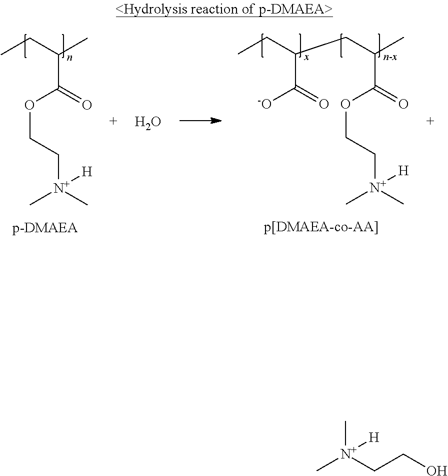

In the above formula, n and x may be natural numbers, and x is smaller than n.

The hydrolyzed polymer 20′ may have a negative charge due to —COO— of an acrylic acid repeating unit. Accordingly, when hydrolysis proceeds, the nucleic acid 10 may be released by electrostatic repulsion between the negatively charged nucleic acid 10 and the negatively charged polymer 20′.

The hydrolysis may be promoted by heat. However, the hydrolysis may be performed at a temperature much lower than that of conventional heat treatment required for releasing nucleic acids, and may release a larger amount of nucleic acids at a faster rate. Thus, the speed, efficiency, and accuracy of the nucleic acid extraction step and the detection method including the same may be enhanced.

For example, a nucleic acid release step by the hydrolysis may be performed at 30° C. to 60° C. or 40° C. to 60° C. When a temperature of the hydrolysis excessively increases, it may affect a subsequent amplification step, and the like, and when the temperature is excessively low, the hydrolysis efficiency and the hydrolysis rate may be greatly lowered.

According to an embodiment, the nucleic acid release step may lead to an amplification step and in-situ. For example, the nucleic acid release step may be performed after adding a PCR mixture to the container for extracting nucleic acid. The polymer may not substantially affect the amplification process. Accordingly, it may be possible to continuously or simultaneously perform the nucleic acid release step and the amplification step without a process for separating the polymer. However, embodiments of the present disclosure are not limited thereto, and the nucleic acid release step may be performed using a separate buffer, and the amplification step may be performed after removing the polymer which releases nucleic acids.

For example, the PCR mixture may include primers, insertion dyes, probes, dNTP which is a mixture of dATP, dTTP, dGTP and dCTP, reverse transcriptase, and the like.

For example, the nucleic acid release step may be performed in an environment of pH 6 to 9 or pH 7.5 to 8.5, and may be performed for one minute to several hours. For example, the hydrolysis efficiency (%) of the polymer may be 30% to 100% after reaction at 50° C. for one hour, preferably may be 30% or more after reaction at 50° C. for 30 minutes, and more preferably may be 30% or more after reaction at 50° C. for five minutes.

After the nucleic acid release step, a polymerase chain reaction may be performed in-situ. For example, the PCR for the amplification step may include at least one of assembly-PCR, asymmetric PCR, digital PCR, endpoint PCR, inverse PCR, methylation-specific PCR, qualitative PCR, quantitative PCR, real-time PCR, and reverse transcription (RT)-PCR, but the embodiments of the present disclosure are not limited thereto, and may be combined with other technologies capable of amplifying nucleic acids in addition to PCR.

Then, the amplified product may be analyzed to detect a target nucleic acid. For example, in order to detect the target nucleic acid, fluorescence derived from the amplified product may be measured.

According to embodiments of the present disclosure, a region for polymer-nucleic acid interaction may be expanded by capturing nucleic acids using a dissolved cationic polymer. Thus, nucleic acid capturing efficiency may be enhanced.

In addition, the nucleic acids may be released by electrostatic repulsion between the polymer and the nucleic acids generated by the self-catalyzed hydrolysis of the polymer. Thus, more nucleic acids may be released at a temperature lower than that of a conventional method for releasing nucleic acids, without changing conditions which may affect an amplification step.

In addition, the polymer may have stability in the amplification step subsequent to a nucleic acid extraction step. Accordingly, it may be possible to implement a detection system for continuously performing the nucleic acid extraction step and the amplification step.

Hereinafter, synthesis and effects of the polymer thin film according to exemplary embodiments will be described in detail through specific experimental examples. However, the experimental examples are provided only by way of example, and the scope of the present disclosure is not limited to the contents provided in the experimental examples.

### EXAMPLE 1

A silicon wafer substrate was placed in an iCVD chamber and a temperature of the substrate was maintained between 25° C. and 40° C. Then, DMAEA and TBPO as an initiator were vaporized at a feed ratio of 80:30 mtorr while maintaining the temperature at 35° C. and 25° C., respectively, and then transferred into a chemical vapor deposition chamber. A pressure in the chamber was maintained at 150 mTorr vacuum state, and at the same time, a filament was heated to 140° C. to radically polymerize the monomers adsorbed on the substrate, thereby preparing a DMAEA homopolymer (thin film thickness: about 350 nm).

### EXAMPLE 2

A silicon wafer substrate was placed in an iCVD chamber and a temperature of the substrate was maintained between 25° C. and 40° C. Then, DMAEA, V3D3 and TBPO were vaporized at a ratio of 150:15:30 mtorr while maintaining the temperature at 35° C., 40° C. and 25° C., respectively, and then transferred into a chemical vapor deposition chamber. A pressure in the chamber was maintained at 250 mTorr vacuum state, and at the same time, a filament was heated to 140° C. to radically polymerize the monomers adsorbed on the substrate, thereby preparing a DMAEA-V3D3 copolymer (thin film thickness: about 350 nm).

### EXAMPLE 3

Radical polymerization was performed in the same manner as in Example 2, except that DMAEA, V3D3 and TBPO were vaporized at a feed ratio of 150:30:30 mtorr, so as to prepare a DMAEA-V3D3 copolymer (thin film thickness: about 350 nm).

FIG. 3 is a graph showing the results of Fourier transform infrared (FTIR) analysis of the DMAEA polymer (pDMAEA) and the monomer (DMAEA) of Example 1.

Referring to FIG. 3, a peak of 1626 cm⁻¹ indicating a vinyl group was smaller in the polymer (pDMAEA) than in the monomer (DMAEA). Thus, it can be confirmed that the polymer is well synthesized (when the polymerization proceeds, the vinyl group of the monomer disappears).

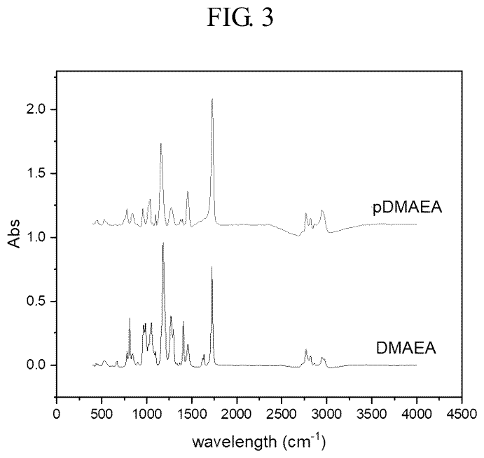

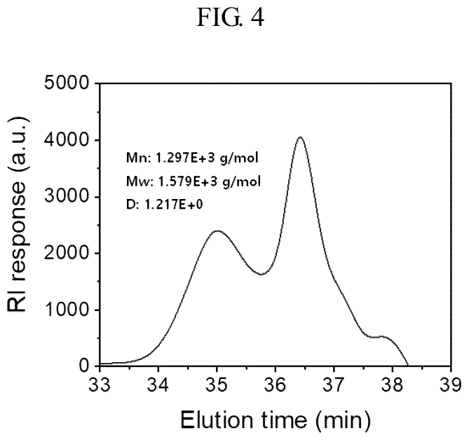

FIG. 4 is a graph showing the results of a gel permeation chromatography (GPC) analysis of a molecular weight of the DMAEA polymer of Example 1. Referring to FIG. 4, a number average molecular weight (Mn) of the DMAEA polymer of Example 1 was about 1,300 g/mol.

### Hydrolysis Experiment

The DMAEA polymer of Example 1 was subjected to hydrolysis at pH 8.0, 25° C. and 50° C. to calculate the hydrolysis efficiency over time (five-minute intervals). Specifically, the hydrolysis efficiency was calculated by comparing a sum of CH2O-methylene signals corresponding to a side chain group which was not hydrolyzed and a sum of the alcohol signals corresponding to reaction by-products using nuclear magnetic resonance (NMR) spectroscopy.

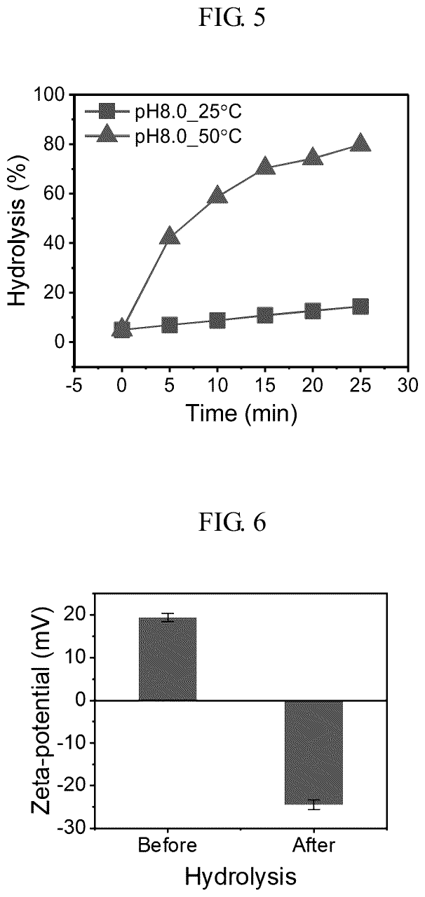

FIG. 5 is a graph showing hydrolysis efficiency (%) over times (five-minute intervals) by subjecting the DMAEA polymer of Example 1 to hydrolysis at pH 8.0, 25° C. and 50° C. Referring to FIG. 5, it can be seen that the hydrolysis of the DMAEA polymer of Example 1 may actively proceed at a temperature of 30° C. or higher, and it was confirmed that the hydrolysis efficiency is 80% or more at 50° C. in 25 minutes later. In particular, it can be confirmed that the hydrolysis of 45% or more proceeds at 50° C. within five minutes.

FIG. 6 is a graph showing a zeta-potential before and after hydrolysis of the DMAEA polymer of Example 1. Referring to FIG. 6, the zeta-potential of the DMAEA polymer of Example 1 was 20 mV before hydrolysis, but changed to −25 mV after hydrolysis, which may be due to an increase in an AA group having a negative charge by hydrolysis.

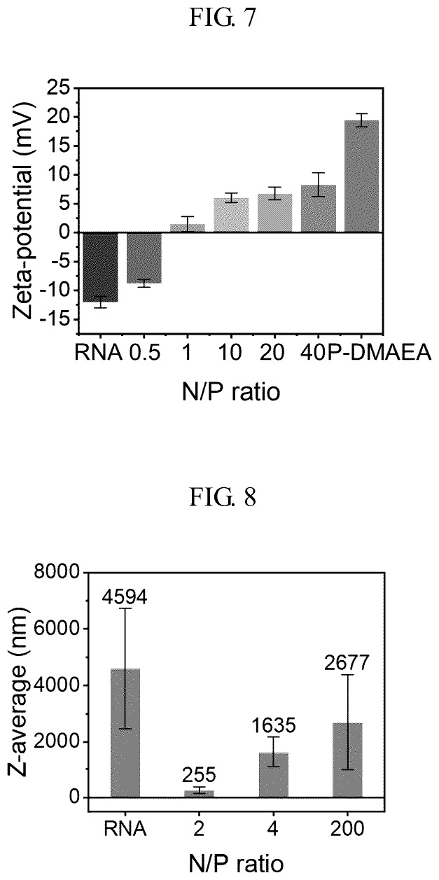

FIG. 7 is a graph showing a zeta-potential according to a ratio of DMAEA polymer of Example 1 and RNA (nitrogen (N)/phosphate (P)). Referring to FIG. 7, it can be confirmed that as the ratio of the DMAEA polymer increases, the zeta-potential value increases, and when the N/P ratio is 1, the zeta-potential value rapidly changes to have a positive value (+).

FIG. 8 is a graph showing an average hydrodynamic diameter (Z-average) according to a ratio of DMAEA polymer and RNA of Example 1 (nitrogen (N)/phosphate (P)). Referring to FIG. 8, it can be confirmed that when N/P ratio is 2, a Z-average value is rapidly decreased to 255 nm and thus a polyplex is formed.

### Nucleic Acid Capturing Experiment

After 3 uL (concentration: 50, 20, 10, 1 ng/uL) of RNA solution (influenza virus RNA, Korea Research Institute of Bioscience and Biotechnology) was added to a tube, in which a substrate coated with the polymer obtained in Examples 1 to 3 was placed, the resulting mixture was centrifuged at room temperature for 1 to 15 minutes to precipitate a polyplex produced by the combination of RNA and DMAEA. The unsettled supernatant was collected, and an amount of RNA in the supernatant was quantified through qRT-PCR, and compared with an initial input amount, so as to calculate the capture efficiency (the amount of RNA in 1-supernatant/initial input amount, %).

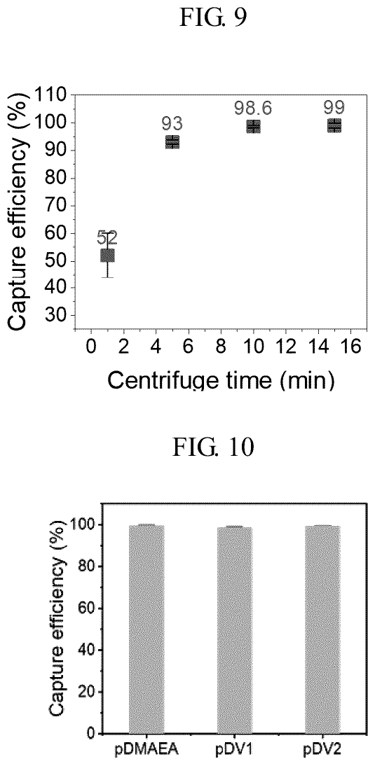

FIG. 9 is a graph showing the capture efficiency according to a centrifugation time in the nucleic acid capturing experiment using the DMAEA polymer of Example 1. Referring to FIG. 9, it was confirmed that 90% or more of nucleic acids may be captured within five minutes.

FIG. 10 is a graph showing capture efficiency during a centrifugation time optimized in a nucleic acid capturing experiment using the polymers of Example 1 (pDMAEA), Example 2 (pDV1), and Example 3 (pDV2). Referring to FIG. 10, the polymers of Example 1 (pDMAEA), Example 2 (pDV1) and Example 3 (pDV2) were used to achieve high capture efficiency close to 100%.

### Nucleic Acid Release Experiment

After 3 uL (concentration: 50, 20, 10, 1 ng/uL) of RNA solution (influenza virus RNA, Korea Research Institute of Bioscience and Biotechnology) was added to a tube, in which a substrate coated with the polymer obtained in Examples 1 to 3 was placed, the resulting mixture was centrifuged at room temperature for five minutes to precipitate a polyplex produced by the combination of RNA and DMAEA. A qRT-PCR mixture was added to the precipitated polyplex and qRT-PCR was performed. In the qRT-PCR process, reverse transcription (RT) as a first step was performed at 50° C. for 5 to 30 minutes. Complementary DNA (cDNA) synthesized through RT (nucleic acidrelease by hydrolysis and RT simultaneous progress) was amplified through a conventional PCR process.

A RNA recovery rate (quantified RNA/captured RNA, %) was calculated by comparing an amount of RNA quantified through qRT-PCR with an amount of captured RNA obtained through RNA capture experiment.

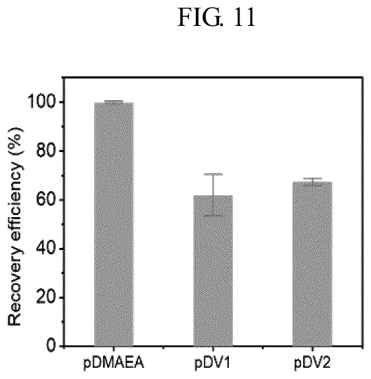

FIG. 11 is a graph showing the recovery efficiency in a nucleic acid extraction experiment using the polymers of Example 1 (pDMAEA), Example 2 (pDV1), and Example 3 (pDV2). In the case of Example 1 (pDMAEA) forming a homopolymer, the recovery rate (close to 100%), which was higher than the recovery rate (70%) of Example 2 (pDV1) and Example 3 (pDV2) forming a cross-linked copolymer, was achieved.

The polymer thin film and the nucleic acid extraction tube according to the exemplary embodiments of the present disclosure may be used in various nucleic acid extraction methods using nucleic acids, and target detection, molecular diagnosis and the like using the same. For example, the polymer thin film and the nucleic acid extraction tube may be used for detection and quantification of pathogens such as viruses, bacteria, etc., investigation of origin of food materials, diagnosis and monitoring of diseases, etc.

Although the foregoing has been described with reference to exemplary embodiments of the present disclosure, those skilled in the art will understand that various modifications and changes can be made within the scope of the present disclosure described in the patent claims below without departing from the spirit and scope of the present disclosure.

What is claimed is:

1. A polymer thin film for extracting nucleic acid comprising a cationic polymer having a hydrolyzable side chain, wherein the polymer thin film has a surface potential of 1 mV to 50 mV, wherein a hydrolysis efficiency of the cationic polymer is 30% or more after reaction at 50° C. for one hour.

2. The polymer thin film of claim 1, wherein the hydrolyzable side chain comprises a hydrolytic cationic ester.

3. The polymer thin film of claim 2, wherein the cationic polymer comprises a repeating unit polymerized from a monomer including at least one of 2-(dimethylamino)ethyl acrylate (DMAEA), 2-(dimethylamino)ethyl methacrylate (DMAEMA), and 2-(diethylamino)ethyl methacrylate (DEAEMA).

4. The polymer thin film of claim 2, wherein the cationic polymer comprises a first repeating unit polymerized from a first monomer including the hydrolytic cationic ester and a second repeating unit polymerized from a second monomer different from the first monomer, and
the second monomer includes at least one of 4-vinylpyridine (4VP), vinylimidazole (VIDZ), dimethylaminomethylstyrene (DMAMS), vinylbenzylchloride (VBC), chloroethylacrylate (CEA), divinylbenzene (DVB), glycidylmethacrylate (GMA), di(ethyleneglycol)divinylether (DEGDVE), ethyleneglycoldimethacrylate (EGDMA), ethyleneglycoldiacrylate (EGDA), 1,4-butadienedioldiacrylate (BDDA), 2,4,6,8-tetramethyl-2,4,6,8-tetravinylcyclotetrasiloxane (V4D4), 1,3,5-trimethyl-1,3,5-trivinyl-cyclotrisiloxane (V3D3), and cyclohexyl methacrylate (CHMA).

5. The polymer thin film of claim 4, wherein a molar ratio of the first repeating unit and the second repeating unit is 1:0.1 to 1:0.3.

6. The polymer thin film of claim 2, wherein the cationic polymer comprises a repeating unit polymerized from DMAEA.

7. The polymer thin film of claim 6, wherein the cationic polymer is a linear polymer.

8. The polymer thin film of claim 6, wherein the cationic polymer has a branched or cross-linked structure.

9. The polymer thin film of claim 1, wherein the cationic polymer has a number average molecular weight of 1,000 g/mol to 3,000 g/mol.

10. A method for extracting nucleic acid, the method comprising:
dissolving a cationic polymer having a hydrolyzable side chain to combine the dissolved cationic polymer with nucleic acid by electrostatic attraction;
precipitating a polyplex formed by combination of the cationic polymer with the nucleic acid; and
hydrolyzing the cationic polymer to release the nucleic acid from the polyplex.

11. The method of claim 10, wherein hydrolyzing the cationic polymer to release the nucleic acid is performed at 30° C. to 60° C.

12. The method of claim 11, wherein the cationic polymer is combined with the nucleic acid at a temperature of 10° C. or more and less than 30° C.

13. The method of claim 10, wherein the hydrolyzed side chain of the cationic polymer has a negative charge.

14. The method of claim 10, wherein a hydrolysis efficiency of the cationic polymer is 30% or more after reaction at 50° C. for one hour.

15. The method of claim 10, wherein the cationic polymer is a linear polymer comprising a repeating unit polymerized from DMAEA.

16. The method of claim 10, wherein the hydrolyzable side chain comprises a hydrolytic cationic ester, and the cationic polymer has a number average molecular weight of 1,000 g/mol to 3,000 g/mol.
